(19) United States
(12) Patent Application Publication
(10) Pub. No.: US 2015/0190451 A1
(43) Pub. Date: Jul. 9, 2015

(54) Herbal Composition and a Method for the Treatment of Hyperlipidemia Caused by Excessive Consumption of Lamb

(71) Applicant: Shuai Dong, Zhangqiu (CN)

(72) Inventor: Shuai Dong, Zhangqiu (CN)

(21) Appl. No.: 14/585,616

(22) Filed: Dec. 30, 2014

(30) Foreign Application Priority Data
Jan. 5, 2014 (CN) 201410002825.7

(51) Int. Cl. A61K 36/88; A61K 36/315; A61K 36/47; A61K 36/28; A61K 36/489; A61K 36/344; A61K 36/00; A61K 36/70; A61K 36/07; A61K 36/71; A61K 36/185; A61K 36/85; A61K 36/41; A61K 36/81; A61K 36/33; A61K 36/60; A61K 36/24; A61K 36/37; A61K 36/748; A61K 36/808; A61K 36/634; A61K 36/65; A61K 35/57; A61K 36/23; A61K 36/484; A61K 36/29

(52) U.S. Cl. CPC A61K 36/88; A61K 36/29; A61K 36/315; A61K 36/47; A61K 36/28; A61K 36/489; A61K 36/344; A61K 36/00; A61K 36/70; A61K 36/07; A61K 36/71; A61K 36/185; A61K 36/85; A61K 36/41; A61K 36/81; A61K 36/33; A61K 36/60; A61K 36/24; A61K 36/37; A61K 36/748; A61K 36/808; A61K 36/634; A61K 36/65; A61K 35/57; A61K 36/23; A61K 36/484

(57) ABSTRACT

A composition and a method for treating hyperlipidemia caused by excessive consumption of lamb. The composition comprises in various parts: root of anhwei barberry, folium isatidis, garden erphorbia herb, aster ageratoides, Vietnamese sophora root, codonopsis ianceolata, all-grass of snakefoot clubmoss, apanese stephania root, amur barberry root, thymifoious euphorbia herb, puff-ball, meadowrue root and rhizome, cottonrose hibiscus flower, common lantana leaf, herba orostachyos, Japanese dock root, pubescent holly root, Chinese brake herb, fruit of tree-of-heaven ailanthus, Chinese iris seed, European verbena herb, datura seed, wild buckwheat rhizome, filliform cassytha herb, fig, radix cynanchi atrati, bittersweet herb, Chinese pulsatilla root, spreading hedyotis herb, figwort root, herb of hygrometric boea, radix ardisiae crenatae, sowthistle tasselflower herb, weeping forsythia, roughhaired holly root, tree peony bark, membrane of chicken gizzard, carrot, medicated leaven, and radix glycyrrhizae. The method comprises weighing each herb, blending them into water and soak, boiling the mixture, and filtering the residue.

The present application hereby claims priority to Chinese application No. 201410002825.7, filed on Jan. 5, 2014.

### TECHNICAL FIELD

The present invention relates to the technical field of a medical composition and a method for medical preparation, and more specifically speaking, it is a preparation for, and a method for the treatment of hyperlipidemia caused by excessive consumption of lamb.

### BACKGROUND OF THE INVENTION

The name of “hyperlipidemia” does not exist in traditional Chinese medicine (“TCM”), but it has long been recognized and referred as “phlegm,” “fat,” “stroke,” “vertigo,” and other diseases under the “dirty blood disease” category. According to TCM, “dirty” blood is loaded with toxins or waste products, which is caused by turbid Qi, phlegm dampness and stasis in blood. It is recorded in Confucian's Duties To their Parents that, “people who eat fine food such as fatty meats and refined grains, will be harmed by food and drinks they consumed, and may develop flatulence, fullness and heartburn.” TCM believes that one of the causes of this disease is patients' excessive consumption of lamb, weight gain, dysfunction of spleen, and the disruption of lipid's transportation, usage, and excretion within human bodies.

Nowadays, lipid regulating drugs that are generally used for the treatment of hyperlipidemia caused by excessive consumption of lamb are listed as follows:
1. Gemfibrozil: The most common side effect is gastrointestinal discomfort, such as indigestion, anorexia, nausea, vomiting, fullness, and stomach discomfort. Patients with known allergic reactions to this drug, or patients who suffer from gallbladder disease, cholelithiasis, liver dysfunction or primary biliary cirrhosis, severe renal insufficiency, and reduction of serum proteins caused by nephrotic syndrome are forbidden to use the product. The product also interferes with the diagnosis, which may lead to a reduction of hemoglobin, hematocrit, or white blood cells, or an elevation of blood creatine phosphokinase, alkaline phosphatase, aminotransferase, or lactate dehydrogenase. After discontinuing the use of the product, the blood cholesterol and triglyceride levels may rebound over the original levels, so that a low-fat diet shall be provided and blood lipids shall be monitored until they reach normal levels.
2. Acipimox: Initial treatment can cause skin vasodilation, increase sensitivity to heat, such as hot flashes over the face or itching all over the body which would usually disappear in a few days after treatment, without stopping the medication. It shall not be used in patients who are allergic to this drug, or who have gastrointestinal ulcers. Pregnant women, lactating women, or children are forbidden to use this drug. Before using this medicine, patients should have low-cholesterol and low-fat diets and quit drinking Dosage shall be reduced in patients with renal insufficiency.
3. Bezafibrate: The most common adverse reactions were gastrointestinal discomfort, such as indigestion, anorexia, nausea, vomiting, and a sense of fullness. It shall not be used in patients who are allergic to the drug, or in patients who suffer from gallbladder diseases, cholelithiasis, hepatic insufficiency or primary biliary cirrhosis, severe renal insufficiency, and hypoalbuminemia caused by nephrotic syndrome. It shall be used with caution in pregnant and lactating women.
4. Fenofibrate: The probability of occurrence of adverse reactions is about 2% -15%, among which gastrointestinal reactions and insomnia are the common reactions. Contraindications include patients with known allergic reactions to this drug, a history of gallbladder disease, cholelithiasis, severe renal insufficiency, hepatic dysfunction, primary biliary cirrhosis, or unexplained chronic liver function abnormalities. It shall be used with caution in patients with gallstone, liver dysfunction and renal insufficiency. Patients need to stop using the product if serum amylase, or blood cholesterol, low-density lipoprotein level elevates while using. This product is not a substitute for diet therapy, and diet control is still necessary. If the product has to be used in combination with hydroxy methyl coenzyme A (HMG-CoA) reductase inhibitors, the patients shall be closely monitored for serum creatine kinase (CK) levels. In case of significantly increased CK values or suspected myopathy or rhabdomyolysis, the usage of the product shall be immediately stopped. Patients shall be informed to consult a doctor immediately when they experience muscle pain, tenderness, or muscle weakness because sometimes they lead to very serious consequences.
5. Fluvastatin: Adverse reaction is a mild and transient indigestion. Patients who suffer active hepatitis or unexplained persistent elevation in serum transaminase, pregnant and lactating women, as well as patients under 18 years old are forbidden to use this product. Alcoholics, patients with past history of liver diseases, and those who are allergic to other HMG-CoA reductase inhibitors are suggested to use the product with caution. When this product is used for treatment, diet therapy must be carried out simultaneously. In case of persistent elevation in serum transaminases, and more than three times over the upper limit, or significantly increased creatine kinase or myositis, this drug shall be discontinued immediately.
6. Alginic Sodium Diester: The probability of occurrence of adverse reactions is 5%-23%, which mainly are palpitations, angina pectoris, hypotensions and electrocardiography (ECG) abnormalities. Possible adverse reactions also include xerostomia, nausea, vomiting, diarrhea, abdominal pain, constipation and loss of appetite. In some cases, patients may also develop liver abnormalities manifested as elevation in thymol turbidity test (TTT) or serum alanine aminotransferase test (ALT), skin redness, itching, rash, annular erythema and erythroderma, extremity venous varix, limb peripheral edema, acute laryngeal edema and anaphylactic shock. It shall not be used in patients with bleeding disorder or bleeding tendency, severe liver and kidney dysfunction. It shall be used with caution in patients with hypotension, hypovolemia, thrombocytopenia, non hyperviscosity syndrome, non-platelet aggregation hyperactivity, and allergic constitution.
7. Lovastatin: The most common side effect is gastrointestinal discomfort, such as, diarrhea and flatulence, and also includes headaches, rashes, dizziness, blurred vision and taste disorders. It can cause reversible elevated serum aminotransferase. Hence it is necessary to monitor patients' liver functions. Patients with known allergic reactions to this product, or patients who have active hepatitis or unexplained persistent elevation in serum aminotransferase are forbidden to use the product. Patients who are allergic to other HMG-CoA reductase inhibitors should use this product with caution. In case of an elevation of serum aminotransferase more than three times over the upper limit while using the product, or a significant increase of creatine kinase or myositis and pancreatitis, patients shall discontinue the usage immediately. When using this product, if patients experience hypotension, severe acute infection, trauma or metabolic disorders, it shall be noted that renal failure may occur secondary to myolysis.
8. Pravastatin: adverse reactions include mildly elevated aminotransferase, rash, myalgia, headache, chest pain, nausea, vomiting, diarrhea, fatigue and so on. Patients with known allergic reactions to this drug, or patients who have active hepatitis or persistent elevation in liver function tests, as well as pregnant and lactating women are forbidden to use the drug. Patients with a history of liver disease or drinking history, as well as patients who are allergic to other HMG-CoA reductase inhibitors shall use the product with caution. It has poor efficacy for treating patients who have homozygous familial hypercholesterolemia. During treatment, liver function should be checked regularly. If persistent ALT or aspartate transaminase (AST) elevation equals or exceeds three times over the upper limit, treatment shall be discontinued. Use of HMG-CoA reductase inhibitor type of lipid-lowering drugs may occasionally cause elevated creatine phosphokinase (CPK). If the elevated value of CPK is ten times over the three times of the normal upper limit, the usage of the product shall be discontinued. In case of unexplained muscle pain, tenderness, and weakness, especially accompanied by malaise and fever, attention should be drawn. In case of hypotension, severe acute infection, trauma and metabolic disorders when using this product, it shall be noted that renal failure may occur secondary to myolysis.
9. Simvastatin is generally well tolerated with mild and transient adverse reactions in most patients. It is prohibited to be used by patients who are allergic to any component, pregnant women, or patients with active hepatitis or unexplained persistent elevation in serum aminotransferase. It shall be used with caution if used in combination with tetrahydronaphthol type of calcium channel blocker Mibefradil. It shall be used with caution in patients who consume excessive alcohol or have a previous history of liver diseases. The conditions of diffused muscle pain, muscle weakness and/or significant CK elevation (more than ten times greater than normal value) should be considered to be myopathy, and the treatment shall be immediately discontinued. For patients with acute or serious conditions suggestive of myopathy and a tendency of acute renal failure caused by rhabdomyolysis, HMG-CoA reductase inhibitor treatment shall be discontinued. Due to complete lack of low-density lipoprotein (LDL) receptors in patients with homozygous familial hypercholesterolemia, the effect of simvastatin treatment is not ideal for such patients.
10. Vitamin E nicotinate: Adverse reactions may include warm feelings on the neck and facial area, skin redness, headaches and other reactions. Patients may also experience serious skin flushing, itching and gastrointestinal discomfort. Patients with arterial bleeding, diabetes, glaucoma, gout, hyperuricemia, liver diseases, ulcer diseases, and hypotension should use the product with caution.

The adverse effects of the above treatments, and the usage limitations in certain group of patients are overcome by the present invention.

### SUMMARY OF THE INVENTION

To overcome the aforementioned shortcomings, the present invention is to provide a preparation and a method for the treatment for hyperlipidemia caused by excessive consumption of lamb. Here, hyperlipidemia refers to lipid metabolism disorders, abnormal fat metabolism or transport, and too high lipid concentration in plasma, including total cholesterol and triglyceride levels higher than those of normal people of the same age. The clinical conditions of hyperlipidemia caused by excessive consumption of lamb are including but not limited to excessive consumption of lamb, overweight, dizziness, shortness of breath, tinnitus, hot flashes, constipation, and yellow-coated tongue.

Unlike Western medicines, TCM formulas comprise many components, usually extracts of raw herbs with each one present in very small quantity. The belief is that there is synergism among different components. One advantage of this kind of formulation is to avoid excessive toxicity when any one component is given in large quantity. The present invention is prepared by forty-six kinds of herbs that are common and can be purchased in any TCM pharmacy. The finished liquid medicine can be easily made and has small side effects, with a shorter course of treatment and a higher cure rate. These herbs assist and guide each other, and could avoid the adverse effects, allergic reactions and toxicity caused by antibiotics in Western medicine.

To obtain the forgoing object, the technical solution is provided as follows. A Chinese medicine preparation and a method for treatment of hyperlipidemia caused by excessive consumption of lamb comprises in one embodiment by weight: 6 parts of root of anhwei barberry, 5 parts of folium isatidis, 6 parts of garden erphorbia herb, 4 parts of aster ageratoides, 5 parts of Vietnamese sophora root, 6 parts of codonopsis ianceolata, 6 parts of all-grass of snakefoot clubmoss, 6 parts of apanese stephania root, 6 parts of amur barberry root, 6 parts of thymifoious euphorbia herb, 6 parts of puff-ball, 3 parts of meadowrue root and rhizome, 6 parts of cottonrose hibiscus flower, 7 parts of common lantana leaf, 6 parts of herba orostachyos, 4 parts of Japanese dock root, 6 parts of pubescent holly root, 2 parts of Chinese brake herb, 6 parts of fruit of tree-of-heaven ailanthus, 4 parts of Chinese iris seed, 6 parts of European verbena herb, 4 parts of datura seed, 6 parts of wild buckwheat rhizome, 2 parts of filliform cassytha herb, 6 parts of fig, 3 parts of radix cynanchi atrati, 6 parts of bittersweet herb, 6 parts of Chinese pulsatilla root, 6 parts of spreading hedyotis herb, 1 parts of figwort root, 6 parts of herb of hygrometric boea, 6 parts of radix ardisiae crenatae, 5 parts of sowthistle tasselflower herb, 6 parts of weeping forsythia, 6 parts of roughhaired holly root, 6 parts of tree peony bark, 4 parts of membrane of chicken gizzard, 6 parts of carrot, 6 parts of medicated leaven, and 9 parts of radix glycyrrhizae.

The processed method of the preparation of TCM for the treatment of hyperlipidemia caused by excessive consumption of lamb comprises the steps as follows: (a) Providing the raw materials as follows: 6-12 parts of root of anhwei barberry, 5-10 parts of folium isatidis, 6-12 parts of garden erphorbia herb, 4-8 parts of aster ageratoides, 5-10 parts of Vietnamese sophora root, 6-12 parts of codonopsis ianceolata, 6-12 parts of all-grass of snakefoot clubmoss, 6-12 parts of apanese stephania root, 6-12 parts of amur barberry root, 6-12 parts of thymifoious euphorbia herb, 6-12 parts of puff-ball, 3-6 parts of meadowrue root and rhizome, 6-12 parts of cottonrose hibiscus flower, 7-14 parts of common lantana leaf, 6-12 parts of herba orostachyos, 4-8 parts of Japanese dock root, 6-12 parts of pubescent holly root, 2-4 parts of Chinese brake herb, 6-12 parts of fruit of tree-of-heaven ailanthus, 4-8 parts of Chinese iris seed, 6-12 parts of European verbena herb, 4-8 parts of datura seed, 6-12 parts of wild buckwheat rhizome, 2-4 parts of filliform cassytha herb, 6-12 parts of fig, 3-6 parts of radix cynanchi atrati, 6-12 parts of bittersweet herb, 6-12 parts of Chinese pulsatilla root, 6-12 parts of spreading hedyotis herb, 1-2 parts of figwort root, 6-12 parts of herb of hygrometric boea, 6-12 parts of radix ardisiae crenatae, 5-10 parts of sowthistle tasselflower herb, 6-12 parts of weeping forsythia, 6-12 parts of roughhaired holly root, 6-12 parts of tree peony bark, 4-8 parts of membrane of chicken gizzard, 6-12 parts of carrot, 6-12 parts of medicated leaven, and 9-18 parts of radix glycyrrhizae; (b) blend the provided raw materials into 1800 ml of water, and soak for 30 minutes; (c) boil the medicinal herbs with gentle heat for 30 minutes; (d) filter the residue to achieve 390 ml of liquid, which is the Chinese medicine for treatment of hyperlipidemia caused by excessive consumption of lamb. When a patient is in need of such treatment, the drug liquid shall be adjusted to 35° C. He or she needs to consume 130 ml of liquid medicine each time, three times a day, and two days for one treatment course. If symptoms still exist, a second course of treatment is required until curing.

Among the forty kinds of herbs, the root of anhwei barberry, folium isatidis, garden erphorbia herb, aster ageratoides, Vietnamese sophora root, codonopsis ianceolata, all-grass of snakefoot clubmoss, apanese stephania root, amur barberry root, thymifoious euphorbia herb, puff-ball, meadowrue root and rhizome, cottonrose hibiscus flower, common lantana leaf, herba orostachyos, Japanese dock root, pubescent holly root, Chinese brake herb, fruit of tree-of-heaven ailanthus, have effects of inducing diuresis for removing edema, speeding up the metabolism and clearing toxic materials, relieving fidgets, and reducing pain. These nineteen herbs play a predominant role in treating hyperlipidemia caused by excessive consumption of lamb, so they are principal drugs.

Chinese iris seed, European verbena herb, datura seed, wild buckwheat rhizome, filliform cassytha herb, fig, radix cynanchi atrati, bittersweet herb, Chinese pulsatilla root, spreading hedyotis herb, figwort root, herb of hygrometric boea, radix ardisiae crenatae, sowthistle tasselflower herb, weeping forsythia, roughhaired holly root, tree peony bark, have the effects of replenishing vital essence, regulating the digestive system, invigorating spleen and normalizing the function of stomach, as well as pain relieving. These eighteen herbs could assist the principal drugs in treatment of hyperlipidemia caused by excessive consumption of lamb, so they are ministerial drugs.

Traditionally, membrane of chicken gizzard, carrot, and medicated leaven is believed to be able to lead the other herbs to diseased tissues, so they are used as an adjuvant drug. Radix glycyrrhizae could relieve spasm and pain, clear away toxic materials, and coordinate the nature of drugs. It is believed to be able to reduce the toxicity of various drugs, so it is used as a guiding drug.

These forty herbs are applied with compatibility, together achieving the effects of removing edema, clearing away heat and toxic materials, reducing dampness jaundice, inducing diuresis for treating stranguria, clearing heat and relieving fidgets, strengthening the spleen and nourishing the stomach, clearing liver and improving vision, nourishing blood to tranquilize mind, invigorating spleen and normalizing the function of stomach as well as pain relieving. With proper compatibility in these herbs, this drug is the best formula for the treatment of hyperlipidemia caused by excessive consumption of lamb.

### EXAMPLES

There are 398 patients with hyperlipidemia caused by excessive consumption of lamb in total, 221 males, accounting for 55.5%, and 177 females, accounting for 44.5%.

TABLE 1
Age range for 398 patients with hyperlipidemia
caused by excessive consumption of lamb

| Age | 28~30 | 31~35 | 36~40 | 41~52 |
|---|---|---|---|---|
| Number of cases | 112 | 138 | 137 | 11 |
| Percentage | 28.1% | 34.7% | 34.4% | 2.8% |

TABLE 2
Efficacy in 369 patients with hyperlipidemia
caused by excessive consumption of lamb

| Efficacy | Cured | Improved | Invalid |
|---|---|---|---|
| Number of cases | [symbol] 95 | 3 | No |
| Percentage | [symbol] 99.2% | 0.8% | No |

[symbol]

indicates data missing or illegible when filed

TABLE 3
Treatment course for 398 patients with hyperlipidemia
caused by excessive consumption of lamb

| Treatment days | 3~5 | 6~9 | 10~14 | 15~21 |
|---|---|---|---|---|
| Number of cases | 125 | 148 | 113 | 12 |
| Percentage | 31.4% | 37.2% | 28.4% | 3% |

TABLE 4
Before and after treatment in 398 patients with hyperlipidemia caused by excessive consumption of lamb

| | | | After treatment | | |
|---|---|---|---|---|---|
| | | Before treatment | Cured | Improved | Invalid |
| Clinical | | Excessive consumption | No excessive | Occasional occasional | Same with |
| conditions | | of lamb, overweight, | consumption of lamb, | dizziness, shortness of | the conditions |
| | | dizziness, shortness of | No dizziness, shortness of | breath, tinnitus, hot | before treatment |
| | | breathe, tinnitus, hot | breath, tinnitus, hot | flashes, constipation, | |
| | | flashes, constipation, | flashes, constipation, | and yellow-coated tongue. | |
| | | and yellow-coated tongue. | and yellow-coated tongue. | | |
| Blood | Total | Increase | Normal | Slight increase | Same with |
| test | cholesterol | | | | the conditions |
| | | | | | before treatment |
| | Triglyceride | Increase | Normal | Slight increase | Same with |
| | | | | | the conditions |
| | | | | | before treatment |

What is claimed is:

1. A composition for treating hyperlipidemia caused by excessive consumption of lamb, wherein the hyperlipidemia refers to lipid metabolism disorders, abnormal fat metabolism or transport, too high lipid concentration in plasma, including total cholesterol and triglyceride levels, higher than those of normal people of the same age, the said composition comprising:
i. root of anhwei barberry in an amount of 6-12 parts by weight,
ii. folium isatidis in an amount of 5-10 parts by weight,
iii. garden erphorbia herb in an amount of 6-12 parts by weight,
iv. aster ageratoides in an amount of 4-8 parts by weight,
v. Vietnamese sophora root in an amount of 5-10 parts by weight,
vi. codonopsis ianceolata in an amount of 6-12 parts by weight,
vii. all-grass of snakefoot clubmoss in an amount of 6-12 parts by weight,
viii. apanese stephania root in an amount of 6-12 parts by weight,
ix. amur barberry root in an amount of 6-12 parts by weight,
x. thymifoious euphorbia herb in an amount of 6-12 parts by weight,
xi. puff-ball in an amount of 6-12 parts by weight,
xii. meadowrue root and rhizome in an amount of 3-6 parts by weight,
xiii. cottonrose hibiscus flower in an amount of 6-12 parts by weight,
xiv. common lantana leaf in an amount of 7-14 parts by weight,
xv. herba orostachyos in an amount of 6-12 parts by weight,
xvi. Japanese dock root in an amount of 4-8 parts by weight,
xvii. pubescent holly root in an amount of 6-12 parts by weight,
xviii. Chinese brake herb in an amount of 2 parts by weight,
xix. fruit of tree-of-heaven ailanthus in an amount of 6 parts by weight,
xx. Chinese iris seed in an amount of 4-8 parts by weight,
xxi. European verbena herb in an amount of 6-12 parts by weight,
xxii. datura seed in an amount of 4-8 parts by weight,
xxiii. wild buckwheat rhizome in an amount of 6-12 parts by weight,
xxiv. filliform cassytha herb in an amount of 2-4 parts by weight,
xxv. fig in an amount of 6-12 parts by weight,
xxvi. radix cynanchi atrati in an amount of 3-6 parts by weight,
xxvii. bittersweet herb in an amount of 6-12 parts by weight,
xxviii. Chinese pulsatilla root in an amount of 6-12 parts by weight,
xxix. spreading hedyotis herb in an amount of 6-12 parts by weight,
xxx. figwort root in an amount of 1-2 parts by weight,
xxxi. herb of hygrometric boea in an amount of 6-12 parts by weight,
xxxii. radix ardisiae crenatae in an amount of 6-12 parts by weight,
xxxiii. sowthistle tasselflower herb in an amount of 5-10 parts by weight,
xxxiv. weeping forsythia in an amount of 6 parts by weight,
xxxv. roughhaired holly root in an amount of 6 parts by weight,
xxxvi. tree peony bark in an amount of 6-12 parts by weight,
xxxvii. membrane of chicken gizzard in an amount of 4-8 parts by weight,
xxxviii. carrot in an amount of 6-12 parts by weight,
xxxix. medicated leaven in an amount of 6-12 parts by weight, and
x1. radix glycyrrhizae in an amount of 9-18 parts by weight.

2. The composition of claim 1, wherein the composition is in a form of a concentrated oral solution.

3. A method for treating hyperlipidemia caused by excessive consumption of lamb, the method comprising administrating an effective amount of a medicinal composition to a subject in need thereof, wherein said medicinal composition is prepared by a method comprising,
i. weighing predetermined amount of each of root of anhwei barberry, folium isatidis, garden erphorbia herb, aster ageratoides, Vietnamese sophora root, codonopsis ianceolata, all-grass of snakefoot clubmoss, apanese stephania root, amur barberry root, thymifoious euphorbia herb, puff-ball, meadowrue root and rhizome, cottonrose hibiscus flower, common lantana leaf, herba orostachyos, Japanese dock root, pubescent holly root, Chinese brake herb, fruit of tree-of-heaven ailanthus, Chinese iris seed, European verbena herb, datura seed, wild buckwheat rhizome, filliform cassytha herb, fig, radix cynanchi atrati, bittersweet herb, Chinese pulsatilla root, spreading hedyotis herb, figwort root, herb of hygrometric boea, radix ardisiae crenatae, sowthistle tasselflower herb, weeping forsythia, roughhaired holly root, tree peony bark, membrane of chicken gizzard, carrot, medicated leaven, and radix glycyrrhizae.
ii. blending the provided raw materials into 1800 ml of water, and soak for 30 minutes;
iii. boiling the medicinal herbs with gentle heat for 30 minutes;
iv. filtering the residue to achieve 390 ml of liquid.
